(19) United States
(12) Patent Application Publication
(10) Pub. No.: US 2021/0260064 A1
(43) Pub. Date: Aug. 26, 2021

(54) POZIOTINIB COMBINATIONS WITH AN ANTI-HER1, HER2 OR HER4 ANTIBODY AND METHODS OF USE THEREOF

(71) Applicants: Spectrum Pharmaceuticals, Inc., Irvine, CA (US); Hanmi Pharmceutical Co., Ltd., Gyeonggi-do (KR)

(72) Inventors: Guru Reddy, Irvine, CA (US); Sunyoung Jang, Gyeonggi-do (KR); Jooyun Byun, Gyeonggi-do (KR)

(21) Appl. No.: 17/250,259

(22) PCT Filed: Jun. 25, 2019
(86) PCT No.: PCT/US19/38974

§ 371 Date: Dec. 22, 2020

Related U.S. Application Data
(60) Provisional application No. 62/689,282, filed on Jun. 25, 2018.
(60) Provisional application No. 62/812,656, filed on Mar. 1, 2019.

(51) Int. Cl. A61K 31/517; A61K 39/395; A61K 31/337; A61K 33/243; A61K 31/513; A61K 31/395; A61K 31/537; A61K 47/68; A61K 9/00; A61P 35/00

(52) U.S. Cl. CPC A61K 31/517; A61K 39/3955; A61K 31/337; A61K 33/243; A61K 31/513; A61K 31/5545; A61K 2039/545; A61K 47/6803; A61K 47/6855; A61K 47/6863; A61K 9/0019; A61K 9/0053; A61P 35/00; A61K 31/537

(57) ABSTRACT

Provided are combinations of poziotinib and an anti-HER1, anti-HER2 or anti-HER4 antibody, optionally with other agents, and use of the combinations for treating cancer.

### CROSS-REFERENCE TO RELATED APPLICATIONS

This application claims the benefit of U.S. Provisional Application No. 62/812,656, filed on Mar. 1, 2019, and U.S. Provisional Application No. 62/689,282, filed on Jun. 25, 2018, the entire disclosures of which are incorporated herein by reference.

### FIELD OF THE DISCLOSURE

The present disclosure relates generally to a method of treating cancer by administering to a subject a pharmaceutical composition comprising poziotinib in combination with an anti-HER1, HER2 or HER4 antibody, optionally further in combination with an additional anticancer drug.

### BRIEF DESCRIPTION OF RELATED TECHNOLOGY

The epidermal growth factor receptor (EGFR) family is known to have four members, i.e., HER1/ErbB1 (commonly referred to as “EGFR”), HER2/ErbB2, HER3/ErbB3, and HER4/ErbB4. EGFRs play an essential role in normal cell regulation through intracellular signal transduction and these proteins regulate cell growth, apoptosis, migration, adhesion, and differentiation. EGFRs are abnormally overexpressed or mutated in most solid tumor cells, and hyperactivation of these receptors triggers a complex, multilayered network of interrelated signaling pathways including downstream up-regulation of the mitogen activated protein kinase (MAPK), phosphoinositide-3-kinase/AKT (PI3K/AKT), and Janus Kinase/Signal Transducer and Activator of Transcription (JAK/STAT) pathways promoting cancer growth, differentiation, angiogenesis, metastasis, and resistance. For example, HER2 is overexpressed in roughly 20% to 25% of breast cancers and is a prognostic marker. HER2-positive breast cancers are characterized as being more clinically aggressive and more invasive than HER2-negative subtypes, are associated with increased growth rates, early systemic metastasis, and poor prognosis. Therefore, blocking the tumor cell signaling pathway mediated by the epidermal growth factor receptor is a desirable target to potentially produce antitumor effects.

Anticancer drugs targeting EGFRs are categorized into two groups: monoclonal antibodies targeting an extracellular domain and small molecule drugs targeting an intracellular tyrosine kinase. The monoclonal antibodies have the advantage of good pharmaceutical efficacy with lower side effects due to selective binding with the epidermal growth factor receptors. However, monoclonal antibodies have drawbacks in that they are expensive and must be administered by injection. Meanwhile, small molecule drugs targeting a tyrosine kinase are relatively inexpensive and orally administrable, and they also have good pharmaceutical efficacy through selectively interacting with the receptor subtypes (e.g., EGFR, HER2, HER3 or HER4), or interacting with multiple receptor subtypes simultaneously.

Ado-trastuzumab emtansine (T-DM1) is an example of a HER2-targeted antibody-drug conjugate where the antibody is the humanized anti-HER2 IgG1, trastuzumab, covalently linked to the microtubule inhibitory drug DM1 (a maytansine derivative) via the stable thioether linker MCC (4-[N-maleimidomethyl] cyclohexane-1-carboxylate). “Emtansine” refers to the MCC-DM1 complex. T-DM1 is indicated as a single agent for treatment of patients with HER2-positive metastatic breast cancer who had previously received trastuzumab and a taxane, either separately or in combination. The structural formula of the trastuzumab-DMI conjugate (T-DM1) is shown below.

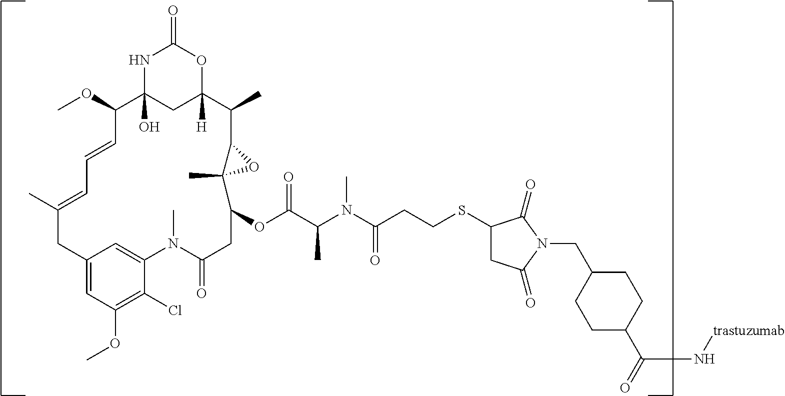

Poziotinib (HM781-36B) is a novel, oral, irreversible pan-HER inhibitor described in U.S, Pat. No. 8,188,102, which is incorporated herein by reference in its entirety. Poziotinib is a quinazoline-based tyrosinase kinase inhibitor (structure shown below) that irreversibly blocks signaling through the EGFR family of tyrosinase-kinase receptors including HER1 (EGFR), HER2, and HER4 wild-type receptors, as well as receptors having activating mutations. This, in turn, leads to inhibition of the proliferation of tumor cells that overexpress these receptors. The administration of poziotinib can lead to the inhibition of the proliferation of tumor cells that overexpress these receptors. The chemical formula of poziotinib is 1-[4-[4-(3,4-dichloro-2-fluorophenylaminino)-7-methoxyquinazolin-6-yloxy]-piperidin-1-yl]prop-2-en-1-one shone below.

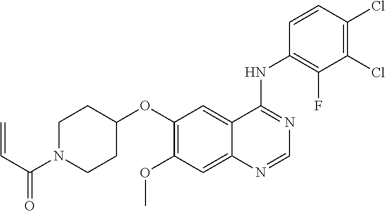 [SMILES: C=CC(=O)N1CCC(Oc2cc3c(Nc4ccc(Cl)c(Cl)c4F)ncnc3cc2OC)CC1]

In recent years, expression of resistance in EGFR target therapy has been reported to decrease response time of the drug used. It has been reported that non-small-cell lung carcinoma (NSCLC) patients having EGFR activating mutations treated with gefitinib or erlotinib are resistant to the drug after about 8 to 16 months of treatment, and about 60% of the patients are observed to be resistant due to the EGFR T790M mutation (Helena A. Yu et al., Clin. Cancer Res. 19(8), 2240, 2013). In addition, in cases of HER2 positive metastatic breast cancer patients treated with the antibody drug, trastuzumab, 66% to 88% of the patients are known to exhibit de novo resistance or acquired resistance due to various mechanisms (Alice Chung et al., Clin. Breast Cancer 13(4), 223, 2013). In this regard, the development of an EGFR-targeting therapeutic agent is limited since its efficacy cannot be maintained for an extended period of time due to generation of primary and secondary resistance, despite the fact that the EGFR targeting therapeutic agent has considerable effect on the treatment of solid cancers with HER2 overexpression or mutation.

Gastric cancer (GC) is the fifth most common cancer and the third leading cause of cancer-related death worldwide. Chemotherapy is the backbone of treatment for most patients with metastatic or recurrent GC. Although there is no universally accepted chemotherapy regimen, a combination of fluoropyrimidine and platinum agents has been most commonly used. Some physicians add docetaxel or epirubicin to this double combination; however, a triple combination is not routinely used because of toxicity concerns.

Therefore, there is an urgent need for an effective therapeutic treatment that can enhance the efficacy and overcome resistance in the treatment of solid cancers having EGFR overexpression or mutation, specifically HER2. The present invention addresses such need.

### SUMMARY

In one aspect, the disclosure provides a method of treating cancer in a subject, the method comprising administering to the subject therapeutically effective amounts of poziotinib and an anti-HER1, HER2, or HER4 antibody, conjugates or fragments thereof where the cancer is associated with overexpression or amplification of HER1, HER2, or HER4, or a mutant of HER1, HER2, or HER4. Suitably the anti-HER2 antibody is trastuzumab or a drug conjugate thereof such as trastuzumab emtansine (T-DM1). The method can further comprise administering at least one agent selected from the group consisting of paclitaxel, cisplatin, 5-fluorouracil, vinorelbine, cetuximab and any combinations thereof. The method is suitable for cancers selected from non-small cell lung cancer, breast cancer, gastric cancer, colon cancer, pancreatic cancer, prostate cancer, myeloma, head and neck cancer, ovarian cancer, esophageal cancer, and metastatic cell carcinoma. Such cancers may be primary or secondary. Suitably the method of treating cancer is directed to a subject having breast cancer or lung cancer or gastric cancer. Suitably the breast cancer is breast cancer including metastatic breast cancer. Suitably the method of treating cancer is directed to a subject having gastric cancer, preferably HER2-positive, more preferably HER2-positive gastric cancer that has previously been treated with one or more chemotherapeutic agents.

In another aspect, the disclosure provides a method of treating breast cancer in a subject in need thereof, wherein the breast cancer is associated with overexpression or amplification of HER1, HER2, HER4 or a mutant of HER2, where the method comprises the steps of a) in a 21-day cycle±3 days, i) administering a single standard dose of anti-HER2 antibody or conjugates thereof such as T-DM1; and ii) administering a daily dose of poziotinib; and b) optionally repeating the cycle. Suitably the T-DM1 is administered by intravenous (IV) infusion. Suitably the T-DM1 standard dose is 0.5 to 10 mg/kg. Suitably T-DM1 is administered at 3.6 mg/kg. Suitably poziotinib is administered orally. Suitably the oral dose of poziotinib is selected from 0.5 to 50 mg/day.

In a further aspect, the disclosure provides a method of improving the adverse events profile in a subject undergoing treatment for breast cancer associated with overexpression or amplification of HER2, or a mutant of HER2, the method comprising the steps of a) in a 21-day cycle±3 days, i) administering a single standard dose of T-DM1; and ii) administering a daily dose of poziotinib; and b) optionally repeating the cycle.

Another aspect of the disclosure provides a combination for treating cancer in a subject, where the cancer is associated with overexpression or amplification of HER1, HER2, or HER4, or a mutant of HER1, HER2, or HER4, and where the combination comprises therapeutically effective amounts of poziotinib and T-DM1, where poziotinib is administered orally and T-DM1 is administered by IV infusion.

Further aspects and advantages will be apparent to those of ordinary skill in the art from a review of the following detailed description. While the methods of treating cancer and the pharmaceutical combinations are susceptible of embodiments in various forms, the description hereafter includes specific embodiments with the understanding that these embodiments are illustrative, and are not intended to limit the invention to the specific embodiments described herein.

### BRIEF DESCRIPTION OF THE DRAWINGS

FIGS. 1A and 1B display the median progression-free survival curve (1A) and the median overall survival curve (1B) for combined Phase I and II patients.

FIGS. 2A and 2B: FIG. 2A displays a Waterfall Plot of Best Percent Change in Tumor Diameter of Target Lesions for combined Phase I and II (2A); FIG. 2B shows the Waterfall Plot for Phase II only.

FIG. 3 displays a Swimmer plot of progression-free survival in the phase II part of Example 3. Duration of Treatment: [(According to the occurrence of event or Date of Last administration)−Date of First administration+1](365.25/52). If the last administration date was not collected, the End of Study Date was used. Best Overall Response (BOR) is presented on the right side of each study duration bar. []: Confirmed BOR. If not specified, BOR and confirmed BOR are the same.

### DETAILED DESCRIPTION

### Definitions

As disclosed herein, a number of ranges of values are provided. It is understood that each intervening value, to the tenth of the unit of the lower limit, between the upper and lower limits of that range is also specifically disclosed, unless the context clearly dictates otherwise. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

As used herein, the term “about” generally refers to plus or minus 10% of the indicated number. For example, “about 10%” may indicate a range of 9% to 11%, and “about 20” may mean from 18 to 22. Other meanings of “about” may be apparent from the context, such as rounding off, so, for example “about 1” may also mean from 0.5 to 1.4. As used herein, the term “and/or” includes any and all combinations of one or more of the associated listed items. Expressions such as “at least one of,” when preceding a list of elements, modify the entire list of elements and do not modify the individual elements of the list. When referring to a dosing protocol, the term “day”, “per day” and the like, refer to a time within one calendar day which begins at midnight and ends at the following midnight.

By the term “treating” or “treatment” and any derivatives thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such a condition or biological manifestation thereof. Prophylactic therapy is appropriate, for example, when the subject is considered at high risk for developing cancer, such as when the subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Specific doses can be readily determined by one having ordinary skill in the art, using routine procedures

By the term “combination” as used herein is meant either simultaneous administration or any manner of separate sequential administration of therapeutically effective amounts of the constituent drugs. Preferably, if the administration is not simultaneous, the compounds are administered in a close time proximity to each other. Suitably, both drugs are administered within about 24, about 12, about 11, about 10, about 9, about 8, about 7, about 6, about 5, about 4, about 3, about 2, or about 1 hour(s) of each other. As used herein, when the administrations of poziotinib and T-DM1 are less than about 45 minutes apart, this is considered to be simultaneous administration.

As used herein, the term “pharmaceutically acceptable carrier and/or excipient” refers to a carrier and/or excipient pharmacologically and/or physiologically compatible to a subject and an active component. A pharmaceutically acceptable carrier includes, without limitation, pH regulators, surfactants, adjuvants, and ionic strength enhancers. For example, pH regulators include, without limitation, phosphate buffer solutions; surfactants include, without limitation, cationic, anionic or nonionic surfactants, for example, Tween-80; ionic strength enhancers include, without limitation, sodium chloride.

As used herein, a “subject in need thereof” refers to a subject or patient suffering from a condition or disease that is associated with overexpression of HER1, HER2 or HER4 or any mutant thereof, who would benefit from the administration of a pharmaceutical combination comprising poziotinib and an anti-HER1, HER2 or HER4 antibody such as trastuzumab or a drug conjugates thereof such as T-DM1. Such subjects particularly include those suffering from HER2-positive breast cancer, or metastatic HER2-positive breast cancer, or HER2-positive gastric cancer. Suitably the cancer may be primary breast cancer, that is, the cancer originated in the breast. Alternatively the cancer may be secondary such as a metastatic breast cancer, that is, the cancer began in the breast and migrated to a secondary site. Suitably the method of treating cancer is directed to a subject having gastric cancer, preferably HER2-positive, more preferably HER2-positive gastric cancer that has previously been treated with one or more chemotherapeutic agents.

The term “wild-type” as used herein is understood in the art and refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild-type polypeptide or polynucleotide, respectively. Included in the term mutant is Single Nucleotide Polymorphism (SNP) where a single base pair distinction exists in the sequence of a nucleic acid strand compared to the most prevalently found (wild-type) nucleic acid strand. Cancers that are either wild-type or mutant for HER1, HER2, or HER4 or have amplification of HER1, HER2, or HER4 genes or have over expression of HER1, HER2, or HER4 protein are identified by known methods.

As used herein, the term “antibody” refers to an immune globulin usually consisting of two pairs of polypeptide chains (each pair has a light (L) chain and a heavy (H) chain). The antibody light chain can be classified as kappa light chain or lambda light chain. The heavy chain can be classified as mu, delta, gamma, alpha or epsilon, and isotypes of the antibody are separately defined as IgM, IgD, IgG, IgA and IgE. In light chain and heavy chain, variable region and constant region are linked via a “J” region with about 12 or more amino acids, and the heavy chain further contains a “D” region having about 3 or more amino acids. Each heavy chain consists of a heavy chain variable region (V H) and heavy constant region (CH). The heavy chain consists of 3 domains (CH1, CH2, and CH3). Each light chain consists of a light chain variable region (VL) and a light chain constant region (CL). The constant regions of the antibody can mediate immune globulin to bind to host tissues or factors, including various cells (e.g., effector cells) of the immune system and first component of the classical complement system.

As used herein, the term “antigen-binding fragment” of an antibody refers to a polypeptide containing a fragment of the full-length antibody, which fragment retains the ability to specifically bind to the same antigen to which the full-length antibody binds, and/or competes with the full-length antibody to specifically bind to the antigen.

### Pharmaceutical Combination

In accordance with the disclosure, the pharmaceutical combination comprises poziotinib and an anti-HER1, anti-HER2 or HER4 antibody or antigenic fragments thereof. Suitably the antibody is selected from the group consisting of trastuzumab, cetuximab, a HER 4 antibody, MA1-861, HFR1, H4.77.16, antigenic fragments or conjugates thereof. Suitably the antibody is trastuzumab, cetuximab or trastuzumab emtansine (T-DM1) and comprises poziotinib or any pharmaceutically acceptable salt thereof. The pharmaceutically acceptable salt may include, without limitation, an acid-addition salt of an inorganic or organic acid. Examples of the inorganic acid addition salt may include salts of hydrochloric acid, hydrobromic acid, sulfuric acid, disulfuric acid, nitric acid, phosphoric acid, perchloric acid, or bromic acid; examples of the organic acid addition salt may include salts of formic acid, acetic acid, propionic acid, oxalic acid, succinic acid, benzoic acid, citric acid, maleic acid, malonic acid, malic acid, tartaric acid, gluconic acid, lactic acid, mandelic acid, glycolic acid, pyruvic acid, glutaric acid, ascorbic acid, palmitic acid, hydroxymaleic acid, hydroxybenzoic acid, phenylacetic acid, cinnamic acid, methanesulfonic acid, benzenesulfonic acid, toluenesulfonic acid, ethanedisulfonic acid, gestisic acid, fumaric acid, lactobionic acid, salicylic acid, phthalic acid, embonic acid, aspartic acid, glutamic acid, camsylic acid, besylic acid, or acetylsalicylic acid (aspirin).

The administration of a therapeutically effective amount of the combinations of the invention are advantageous over the individual component compounds in that the combinations provide one or more of the following improved properties when compared to the individual administration of a therapeutically effective amount of a component compound; i) a greater anticancer effect than the most active single agent, ii) synergistic or highly synergistic anticancer activity, iii) a dosing protocol that provides enhanced anticancer activity with a reduced side effect profile, iv) a reduction in the toxic effect profile, v) an increase in the therapeutic window, vi) an increase in the bioavailability of one or more of the component compounds, or vii) an increase in apoptosis over the individual component compounds.

The present invention also relates to a use of the poziotinib combinations with anti-HER2 antibody or an antigen binding fragment thereof or the conjugate of the poziotinib with such an antibody moiety in manufacturing a kit, wherein the kit is used for detecting the existence of HER2 or the level of HER2 in a sample prior to administration of effective amounts of the combination to the patient. The kit optionally contains instructions for use in detecting the existence of HER2 or the level of HER2 in a patient's biological sample.

### Administration of the Pharmaceutical Combination

Several malignancies, including lung, breast, gastric, colorectal, head, and neck, and pancreatic carcinomas, are associated with a mutation in, or overexpression of, members of the EGFR receptor family. Poziotinib has demonstrated therapeutic activity in treating various such cancers, including lung, gastric, breast, and head and neck cancers.

The present disclosure further provides methods of treating cancer comprising administering to a subject in need thereof the pharmaceutical composition as described herein. Suitably the subject in need thereof is administered a therapeutically effective amount of the pharmaceutical combination of poziotinib and an anti-HER2 antibody that recognizes a HER2 extracellular domain epitope with an antigen affinity constant that can be up to 0.1 nmol/L. Suitably the anti-HER2 antibody is a humanized monoclonal antibody, such as trastuzumab, that recognizes HER2 extracellular domain IV juxtamembrane epitope, and its antigen affinity constant can be up to 0.1 nmol/L. Suitably the antibody recognizes the epitope consisting of the 3 loops (557-561, 570-573 and 593-603) at the C-terminus of section IV. Suitably the antibody may be a humanized bispecific anti-HER2 antibody or a bispecific antigen-binding fragment thereof, comprising one antigen binding site containing variable regions of heavy and light chain of trastuzumab, and another antigen binding site containing variable regions of heavy and light chain of pertuzumab. The bispecific antibody preferably recognizes HER2 extracellular domains IV and II. Suitably the antibody may be a chimeric (mouse/human) monoclonal antibody such as Cetuximab.

Suitably the pharmaceutical combination is a combination of poziotinib and trastuzumab emtansine conjugate (T-DM1). Poziotinib may be administered in an amount of 0.1 mg to 50 mg. T-DM1 may be administered in an amount of 0.5 to 10 mg per kg of patient's body weight. Preferably T-DM1 is administered in an amount of 1.5 to 5.5 mg per kg of body weight. The combination can further comprise oral paclitaxel.

Alternatively the antibody is Cetuximab, an epidermal growth factor receptor (EGFR) inhibitor used for the treatment of metastatic colorectal cancer, metastatic non-small cell lung cancer and head and neck cancer. Cetuximab is a chimeric (mouse/human) monoclonal antibody given by intravenous infusion that is distributed under the trade name Erbitux™ in the U.S. and Canada by the pharmaceutical company Bristol-Myers Squibb and outside the U.S. and Canada by the pharmaceutical company Merck KGaA. In Japan, Merck KGaA, Bristol-Myers Squibb and Eli Lilly have a co-distribution. As a further agent to the poziotinib/T-DM1 combination, cetuximab may be administered in an amount of from 100 mg/m2 to 500 mg/m2 of a surface area of the body.

Vinorelbine (NVB), sold under the brand name Navelbine™, among others, is a chemotherapy medication used to treat a number of types of cancer. This includes breast cancer and non-small cell lung cancer. It is given by intravenous injection or by mouth. Vinorelbine is in the vinca alkaloid family. Without wishing to be bound by any particular theory, vinorelbine is believed to work by disrupting the normal function of microtubules and thereby stopping cell division. As a further agent to the poziotinib/T-DM1 combination, vinorelbine may be administered in an amount of 0.5 mg/m2 to 50 mg/m2 of a surface area of the body.

Paclitaxel (PTX), sold under the brand name Taxol™ among others, is a chemotherapy medication used to treat a number of types of cancer. This includes ovarian cancer, breast cancer, lung cancer, Kaposi sarcoma, cervical cancer, and pancreatic cancer, It is given by intravenous injection. As a further agent to the poziotinib/T-DM1 combination, paclitaxel may be administered in an amount of 100 mg/m2 to 300 mg/m2 of a surface area of the body.

Suitably the combination of poziotinib and T-DM1 can further include a mitotic inhibitor. The mitotic inhibitor may be selected from BT-062, HMN-214, eribulin mesylate, vindesine, EC-1069, EC-1456, EC-531, vintafolide, 2-methoxyestradiol, GTx-230, crolibulin, D1302A-maytansinoid conjugates, IMGN-529, lorvotuzumab mertansine, SAR-3419, SAR-566658, IMP-03138, topotecan/vincristine combinations, BPH-8, fosbretabulin tromethamine, estramustine phosphate sodium, vincristine, vinflunine, vinorelbine, RX-21101, cabazitaxel, STA-9584, vinblastine, epothilone A, patupilone, ixabepilone, Epothilone D, paclitaxel, docetaxel, DJ-927, discodermolide, eleutherobin, and pharmaceutically acceptable salts thereof or combinations thereof. For example, the combination may further include a taxane, a vinca alkaloid, or a combination thereof. The vinca alkaloid may be at least one drug selected from the group consisting of vinblastine, vincristine, vindesine and vinorelbine. The taxane may be paclitaxel or docetaxel. Suitably the combination of poziotinib and T-DM1 may further include paclitaxel or vinorelbine. Preferably the combination of poziotinib and T-DM1 further includes paclitaxel.

Suitably the combination of poziotinib and T-DM1 can further include an mTOR inhibitor. The mTOR inhibitor may be selected from zotarolimus, umirolimus, temsirolimus, sirolimus, sirolimus NanoCrystal™, sirolimus TransDerm™, sirolimus-PNP, everolimus, biolimus A9, ridaforolimus, rapamycin, TCD-10023, DE-109, MS-R001, MS-R002, MS-R003, Perceiva, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, ABTL-0812, and pharmaceutically acceptable salts thereof or combinations thereof. For example, the combination of poziotinib and T-DM1 may further include rapamycin. The rapamycin may be in the form of an injection. Rapamycin, also known as sirolimus, is a compound produced by the bacterium Streptomyces hygroscopicus. Rapamycin may be administered in an amount of 0.5 mg/m2 to 10 mg/m2 of a surface area of the body.

Suitably the combination of poziotinib and T-DM1 may further include an antimetabolite. The antimetabolite may be selected from the group consisting of capecitabine, 5-fluorouracil, gemcitabine, pemetrexed, methotrexate, 6-mercaptopurine, cladribine, cytarabine, doxifludine, floxuridine, fludarabine, hydroxycarbamide, decarbazine, hydroxyurea, and asparaginase. For example, the combination of poziotinib and T-DM1 can further include 5-fluorouracil. The 5-fluorouracil may be in the form of an injection. 5-Fluorouracil may be administered in an amount of 100 mg/m2 to 3,000 mg/m2 of a surface area of the body.

Fluorouracil (5-FU), sold under the brand name Adrucil™ among others, is a pyrimidine analog used to treat cancer. By intravenous injection it is used for treating colon cancer, esophageal cancer, gastric cancer, pancreatic cancer, breast cancer, and cervical cancer. As a topical cream it is used for basal cell carcinoma. Its mechanism of action is not entirely clear, but, without wishing to be bound by any particular theory, it is believed to involve blocking the action of thymidylate synthase and thus stopping the production of DNA.

Suitably the combination of poziotinib and T-DM1 can further include a platinum-based antineoplastic drug. The platinum-based antineoplastic drug may be selected from the group consisting of cisplatin, carboplatin, dicycloplatin, eptaplatin, lobaplatin, miriplatin, nedaplatin, oxaliplatin, picoplatin, and satraplatin. For example, the poziotinib/T-DM1 combination can further include cisplatin. The cisplatin may be in the form of an injection. Cisplatin may be administered in an amount of 1 mg/m2 to 100 mg/m2 of a surface area of the body. Without wishing to be bound by any particular theory, it is believed that cisplatin works in part by binding to DNA and inhibiting DNA replication.

Therapeutically effective amounts of the combinations of the invention are administered to a human. Typically, the therapeutically effective amount of the administered agents of the present invention depend upon a number of factors including, for example, the age and weight of the subject, the precise condition requiring treatment, the severity of the condition, the nature of the formulation, and the route of administration. Ultimately, the therapeutically effective amount will be at the discretion of the attending physician.

Suitably, the compounds are administered either simultaneously or sequentially in each treatment cycle. When not administered simultaneously, they are both administered within about 24, about 12, about 11, about 10, about 9, about 8, about 7, about 6, about 5, about 4, about 3, about 2, or about 1 hour(s) of each other—in this case, the specified time period will be about 24 12, 11, 10, 9, 8, 7, 6, 5, 4, 3, 2, or 1 hour(s). As used herein, for a two-constituent drug combination, such as poziotinib and T-DM1, the administration of poziotinib and T-DM1 less than about 45 minutes apart is considered simultaneous administration.

Suitably, in each treatment cycle both compounds are administered within a specified period for at least 1, at least 2, at least 3, at least 5, at least 7, at least 14, at least 21 or at least 30 day(s)—in this case, the duration of the treatment cycle is at least 1, 2, 3, 5, 7, 14, 21 or 30 day(s). When, during the course of treatment, both compounds are administered within a specified period for more than 30 days, the treatment is considered chronic treatment and continues until an altering event, such as a reassessment in cancer status or a change in the condition of the patient or one or more serious adverse events, warrants a modification to the protocol.

Suitably, during the course of treatment, such as the drug combination of poziotinib and T-DM1, both constituent drugs are administered within a specified time period for at least 1 day, followed by the administration of poziotinib alone for at least an additional 1, 2, 3, 4, 5, 6, 7, 8, 9, 10, 11, 12, 13, 14, 15, 16, 17, 18, 19, 20, 21, 22, 23, 24, 25, 26, 27, 28 or 29 day(s)—thus, in this embodiment the duration of treatment is at least 2 to 30 days, respectively. Suitably the treatment cycle is 21 days±3 days. Suitably the treatment cycle is 21 days.

Also, contemplated herein is a drug holiday, or rest period, between the sequential administration of one of poziotinib and the other constituent drug, and the other drug. As used herein, a drug holiday (rest period) is a period of days after the sequential administration of one of poziotinib and the other constituent drug, and before the administration of the other where neither poziotinib nor the other constituent drug is administered. Suitably the drug holiday is a period of days selected from: 1 day, 2 days, 3 days, 4 days, 5 days, 6 days, 7 days, 8 days, 9 days, 10 days, 11 days, 12 days, 13 days and 14 days.

It is understood that each treatment cycle can be followed by one or more cycles of repeat dosing or can be followed by an alternate dosing protocol, and further that a drug holiday may precede the repeat dosing or alternate dosing protocol.

Where the subject has breast cancer with a HER2 exon 20 mutation, the HER2 exon 20 mutation may suitably comprise a HER2 in-frame exon 20 insertion mutation, a HER2 exon 20 point mutation, or both. The HER2 in-frame exon 20 insertion mutation may be selected from the group consisting of A775_G776insYVMA, G776_V777insVC, P780_Y781insGSP, and combinations thereof. The HER2 exon 20 point mutation may be selected from the group consisting of L775S, G776V, V777L, and combinations thereof. Suitably the HER2 exon 20 mutation is not a T790M point mutation.

Wild-type or mutant HER1, HER2, and HER4 tumor cells can be identified by DNA amplification and sequencing techniques, DNA and RNA detection techniques, including, without limitation, Northern and Southern blot, respectively, and/or various biochip and array technologies or in-situ hybridization. Wild type and mutant polypeptides can be detected by a variety of techniques including, without limitation, immunodiagnostic techniques such as ELISA, Western blot or immunocytochemistry.

Preferably the method comprises administering to a subject in need thereof a pharmaceutical combination comprising poziotinib and T-DM1, and optionally other chemotherapeutic agents. Preferably the optional chemotherapeutic agent comprises paclitaxel.

Suitably, patients are screened for assessment of their HER1, HER2, or HER4 status. It will be understood by the person of skill in the art that the daily dosage amount of the pharmaceutical combinations described herein will be decided by a physician within the scope of sound medical judgment. The specific dose level for any particular patient will depend upon a variety of factors including the cancer being treated and its severity; the activity of the poziotinib as formulated in the pharmaceutical composition; the specific pharmaceutical composition employed; the age, body weight, general health, sex, and diet of the subject in need thereof; the time of administration; the prescribed number of doses per administration; the duration of the treatment; drugs used in combination or coincidental with the specific pharmaceutical composition employed; and like factors well known in the medical arts.

For example, it is within the skill of those trained in the art to start at lower level doses than required to achieve the desired therapeutic effect and to gradually increase the dosage until the desired effect. Suitably, subjects receiving the present combination exhibit an improved toxicity profile as compared to subjects receiving the standard dosing of the either poziotinib or the anti-HER1, anti-HER2 or anti-HER4 antibody when administered individually. Suitably, the improved toxicity relates to reduction in cardiotoxicity such as troponin I increase above the 99th percentile of the upper reference limit (URL) for the normal range of the assay being used, or any Grade 3 cardiovascular toxicity. Suitably, the improved toxicity relates to improvement in hematologic toxicities. For example, the subjects receiving the combination therapy show a statistical improvement in their rate of thrombocytopenia and neutropenia as compared to subjects receiving individual medications.

Suitably, subjects receiving the combination therapy exhibit an improved and favorably reduced rate in developing non-hematological toxicities such as diarrhea, skin rash, mucositis, fatigue, electrolyte or hepatotoxicity profiles.

Another aspect of the invention is directed to a method of treating cancer in a subject, the method comprising administering to the subject therapeutically effective amounts of poziotinib and an anti-HER1, anti-HER2 or anti-HER4 antibody, where the cancer is associated with overexpression or amplification of HER1, HER2, or HER4, or a mutant of HER1, HER2, or HER4. The method can further comprise administering at least one agent selected from the group consisting of paclitaxel, cisplatin, 5-fluorouracil, vinorelbine, cetuximab and any combinations thereof. The antibody is preferably an anti-HER2 antibody selected from the group consisting of trastuzumab, cetuximab, and any antigen-binding fragments thereof. The cancer can be non-small cell lung cancer, breast cancer, gastric cancer, colon cancer, pancreatic cancer, prostate cancer, myeloma, head cancer, neck cancer, ovarian cancer, esophageal cancer, or metastatic cell carcinoma. In at least one embodiment the method of treating cancer is directed to a subject having breast cancer or gastric cancer.

Suitably the target cancer of the method is breast cancer, where the breast cancer can be selected from the group consisting of (i) estrogen receptor-negative breast cancer with overexpression of HER1 and/or HER2; (ii) estrogen receptor- and progesterone receptor-double positive breast cancer with HER2 being expressed but without overexpression thereof; (iii) trastuzumab-resistant breast cancer with overexpression of HER2; and (iv) HER1-overexpressing breast cancer negative with respect to PR, HER2 and estrogen receptor. Suitably the breast cancer is metastatic breast cancer. The method can further comprise the preliminary steps of collecting breast cancer cells from the subject; and evaluating the breast cancer cells to confirm overexpression of HER2, or overexpression of a mutant of HER2, or amplification of the HER2 gene, or amplification of a mutant of the HER2 gene. Suitably the evaluating step comprises immunohistochemistry (IHC) with confirmatory fluorescence in situ hybridization (FISH), where the IHC can be IHC 3+ or IHC 2+.

In one embodiment, the method of treating cancer is directed to a subject having gastric cancer, preferably HER2-positive, and more preferably HER2-positive gastric cancer that has previously been treated with one or more chemotherapeutic agents.

Another aspect of the invention is directed to a method of treating breast cancer in a subject in need thereof, where the breast cancer is associated with overexpression or amplification of HER2, or overexpression or amplification of a mutant of HER2, the method comprising the steps of a) in a 21-day cycle±3 days, i) administering a single dose of T-DM1 ranging from 1.5-5.5 mg/kg; and ii) administering a daily dose of poziotinib ranging from 0.5-50 mg/day; and b) optionally repeating the cycle. Suitably the T-DM1 is administered by intravenous (IV) infusion. Suitably the T-DM1 standard dose is 3.6 mg/kg. Suitably the poziotinib is administered orally, and the oral dose can be selected from the group consisting of 6, 8, 10, 12, 16 and 24 mg once per day.

A further aspect of the invention is directed to a method of improving the adverse events profile in a subject undergoing treatment for breast cancer associated with overexpression or amplification of HER2, or a mutant of HER2, the method comprising the steps of a) in a 21-day cycle±3 days, i) administering a single dose of T-DM1; and ii) administering a daily dose of poziotinib; and b) optionally repeating the cycle, where the adverse effect is selected from the group consisting of cardiotoxicity hematologic toxicities, diarrhea, skin rash, mucositis, fatigue, electrolyte abnormalities and hepatotoxicity.

Another aspect of the invention is directed to a combination for treating cancer in a subject, where the cancer is associated with overexpression or amplification of HER1, HER2, or HER4, or a mutant of HER1, HER2, or HER4, and combination comprises therapeutically effective amounts of poziotinib and T-DM1, where poziotinib is administered orally and T-DM1 is administered by IV infusion. The combination can further comprise a therapeutically effective amount of paclitaxel administered by intravenous (IV) infusion.

A further aspect of the invention is directed to a method of treating gastric cancer in a subject in need thereof, where the gastric cancer is associated with overexpression or amplification of HER2, or overexpression or amplification of a mutant of HER2, and where the method comprises the steps of: a) in a 21-day cycle±3 days, i) administering a single dose of trastuzumab ranging from 6-8 mg/kg; ii) administering a single dose of paclitaxel ranging from 105-175 mg/m²; and iii) administering a daily dose of poziotinib ranging from 4-16 mg/day; and b) optionally repeating the cycle. Preferably the trastuzumab is administered by intravenous (IV) infusion on Day 1. Preferably the trastuzumab standard dose is 8 mg/kg loading, followed by 6 mg/kg infusion. Preferably the paclitaxel is administered by intravenous (IV) infusion on Day 1. Suitably the paclitaxel standard dose is 175 mg/m² infusion. Suitably the poziotinib is administered orally for 14±3 days. Suitably the oral dose of poziotinib is selected from the group consisting of 4, 6, 8, 10, 12, and 16 mg once per day. Suitably a rest period of 7 days follows the 14-day poziotinib administration.

Embodiments will now be described in more detail with reference to the following examples. However, the examples are not intended to limit the scope of the embodiments.

### EXAMPLES

### Example 1: Method of Treatment of HER2-Positive Breast Cancer

Typically, a female patient having breast cancer is identified, baseline imaging is performed, breast tissue is procured for tumor genotyping, the patient undergoes a treatment period based on the tumor typing, the treatment is evaluated and optionally further continued or amended.

The patient is confirmed for HER2 overexpression or a gene-amplified tumor via an immunohistochemistry (IHC) designation of IHC 3+ or IHC 2+, with confirmatory fluorescence in situ hybridization (FISH). The IHC test provides a score of 0 to 3+ that measures the amount of HER2 receptor protein on the surface of cells in a breast cancer tissue sample. If the score is 0 to 1+, the tissue is designated as “HER2 negative.” If the score is 2+, the tissue is designated as “borderline.” A score of 3+ is designated “HER2 positive.”

The patient is treated in 21-day cycles±3 days with a 3.6 mg/kg T-DM1 IV infusion on day 1, and oral poziotinib on days 1-21 in a single dose at a rate selected from 6 gm, 8 mg, 10 mg, 12 mg, or 16 mg per day. A high oral poziotinib dose of 24 mg per day can be administered, but the dosing regimen can include a 1-week rest period, viz. 24 mg/day administered on days 1-14, followed by a rest interval on days 15-21 of the 21-day cycle. These 21-day cycles are continued until disease progression, death, or intolerable adverse events (AE) dictate otherwise.

A Phase 1b, open-label, multicenter study is performed to determine the Maximum Tolerated Dose (MTD) or Maximum Administered Dose (MAD) of poziotinib when administered with standard dosing of T-DM1 in women with advanced or metastatic HER2-positive breast cancer. The poziotinib dose identified in Part 1 of the study is used in combination with the standard starting dose of T-DM1 (3.6 mg/kg IV on Day 1 of each 21-day cycle) in Part 2 of the study for efficacy evaluation.

Thus, Part 1 of the study is for dose-finding. Cohort 1 begins with 8 mg oral poziotinib, in combination with the standard starting dose of T-DM1 (3.6 mg/kg IV) on Day 1 of each cycle and follow with daily oral doses of 8 mg poziotinib. Based on a “3+3” design, poziotinib dose escalation/de-escalation proceeds based on the occurrence of dose-limiting toxicities (DLTs) during Cycle 1 of the current dose cohort.

A DLT is defined as any of the following treatment-related toxicities occurring during the first treatment cycle:
Cardiotoxicity:
   Troponin I increase above the 99th percentile of the upper reference limit (URL) for the normal range of the assay being used or any Grade 3 cardiovascular toxicity
Hematologic toxicities:
   Grade 3 thrombocytopenia with bleeding;
   Grade 4 neutropenia >7 days or Grade 3 febrile neutropenia
Non-hematologic toxicities:
   Grade 3 or greater diarrhea lasting >3 days on medical therapy or associated with fever ≥100.5° F. (38.1° C.) for at least 2 days or severe dehydration;
   Grade 4 skin rash or mucositis;
   Grade ≥3 fatigue ≥1 week;
   Evidence of hepatotoxicity according to Hy's Law (ALT or AST ≥3×ULN with total bilirubin >2×ULN without existing viral hepatitis, liver diseases, or hepatic metastasis; patients who enrolled with liver metastases, ALT or AST >8×ULN or AST or ALT >5×ULN for ≥14 days;
   Grade ≥3 electrolyte abnormality that lasts >72 hours, unless the patient has clinical symptoms, in which case all Grade ÷3 electrolyte abnormalities, regardless of duration, should count as a DLT; Grade ≥3 amylase or lipase elevations NOT associated with symptoms or clinical manifestations of pancreatitis do not need to be counted as a DLT;

Any other Grade ≥3 treatment-related, non-hematologic toxicity;

Any death not clearly due to the underlying disease or extraneous causes.

Toxicity is assessed based on the grade of the adverse events using the National Cancer Institute (NCI) Common Terminology Criteria for Adverse Events (CTCAE) Scale, version 4.03.

In Phase 1b, Part 1, the dose of poziotinib, in combination with the standard starting dose of T-DM1 (3.6 mg/kg IV) on Day 1 of each cycle is determined using a “3+3” design with up to 3 dose levels tested beginning with 8 mg/day. Poziotinib dose escalation/de-escalation for the next dose cohort proceeds based on the occurrence of dose-limiting toxicities (DLTs) during Cycle 1 of current dose cohort. Patients who complete Cycle 1, continue treatment at that cohort dose until discontinuation of therapy. The four possible cohorts includes 8, 10 and 12 mg/day of poziotinib, plus a reduced dose of 6 mg/day poziotinib.

In Part 2 of the Phase 1 b study, approximately 10 patients are treated at the MTD/MAD to confirm dose for safety of the combination and to evaluate preliminary efficacy. Treatment for all patients continues until disease progression, unacceptable toxicity, or continuation of study treatment is not in the best interest of the patient.

During each 21-day cycle, patients who are eligible for participation receive poziotinib at the assigned dose, orally, once daily, continuously at approximately the same time each morning with a glass of water and breakfast. T-DM1 at 3.6 mg/kg IV is administered on Day 1 of each treatment cycle. On days when T-DM1 is given, poziotinib is given sequentially after the end of infusion.

After 3 treatment cycles, tumor response is assessed every 9 weeks±14 days.

Example 2: Method of Treatment of HER2-Positive Breast Cancer—Dose Regimen, Efficacy, and Safety/Tolerability A Phase 2, open-label, multicenter study is used to establish the dose regimen and evaluate the preliminary efficacy and the safety/tolerability of poziotinib in patients with HER2-positive metastatic breast cancer (MBC) who have received at least two prior HER2-directed treatment regimens including trastuzumab and T-DM1. Poziotinib is administered in 21-day treatment cycles at a dose of 24 mg/day (for 2 weeks followed by a 1-week rest period), 16 mg/day (continuous daily dosing) or 12 mg/day (continuous daily dosing). Toxicity is assessed based on the grade of the adverse events using the National Cancer Institute (NCI) Common Terminology Criteria for Adverse Events (CTCAE) Scale, version 4.03.

With regard to combination treatment further including another agent, in the case of breast cancer cells a high synergistic effect has been observed when poziotinib was combined with paclitaxel, cisplatin, or 5-FU in BT-474 cells, which are HER2-overexpressed and negative to an estrogen receptor (ER). When poziotinib was combined with trastuzumab, a weak synergistic effect was exhibited at some concentrations equal to or lower than the GI₅₀ of poziotinib alone, where the GI₅₀ is the concentration of drug that causes 50% reduction in proliferation of the cancer cells. When poziotinib was combined with vinorelbine, a synergistic effect was not observed at concentrations equal to or lower than the GI₅₀ of poziotinib alone. Similarly, a sufficient effect was observed to indicate synergy when vinorelbine was combined with poziotinib in SK-BR-3 cells, which are HER2-overexpressed and ER-negative. However, the combination of poziotinib with vinorelbine exhibited a synergistic effect at some concentrations in MDA-MB-361 cells, which are HER2-overexpressed, ER-positive, as well as trastuzumab resistant, and this combination exhibited synergy at every concentration in MCF-7 cells, which are not HER1 and HER2-overexpressed and ER-positive. Also, a synergistic effect was observed by the combination of poziotinib and vinorelbine at some concentrations in MDA-MB-468 cells, which are HER2 negative, ER-negative, and HER1-overexpressed triple negative breast cancer cells. In MBA-MB-453 cells, which are cells in which HER2 is overexpressed among trastuzumab resistant breast cancer cells, an excellent synergistic effect was observed at concentrations equal to or lower than the GI₅₀ of poziotinib alone when paclitaxel, 5-FU, cisplatin, or trastuzumab was combined with poziotinib.

The synergistic effect of combining poziotinib with other drugs was also excellent when poziotinib was combined with 5-FU in TE cells, which is a HER2-overexpressed esophageal cancer cell line. In addition, when poziotinib was combined with trastuzumab in N-87 cells, which is a HER2-overexpressed gastric cancer cell line, a synergistic effect was observed at some concentrations.

Thus the combination of poziotinib and T-DM1, further comprising other target anticancer agents or cytotoxic anticancer agents, preferably where the cancer is associated with overexpression or amplification of HER1, HER2, or HER4, or a mutant of HER1, HER2, or HER4, would be highly effective in cancers such as breast cancer, gastric cancer, lung cancer, and esophageal cancer, and would effectively inhibit cancer that is resistant to conventional therapeutic agents. Suitably the cancer is HER2-positive breast cancer.

### Example 3: Method of Treatment of HER2-Positive Advanced Gastric Cancer

A prospective phase I/II study was conducted at 11 treatment centers in Korea. Patients with HER2-positive GC who previously had received one or more lines of chemotherapy were enrolled. Enrolled patients received oral poziotinib (8 mg or 12 mg) once daily for 14 days, followed by 7 days off. Paclitaxel (175 mg/m² infusion) and trastuzumab (8 mg/kg loading dose, then 6 mg/kg infusion) were administered concomitantly with poziotinib on day 1 every 3 weeks

### Methods

Patients. Key eligibility criteria included: patients aged ≥19 years; histopathologically-confirmed locally advanced unresectable, relapsed or metastatic gastric adenocarcinoma (including adenocarcinoma of the esophagogastric junction); HER2 immunohistochemistry (IHC) 3+ or HER2 IHC 2+ and HER2 fluorescence in situ hybridization (FISH)+; the presence of one or more measurable lesions by Response Evaluation Criteria in Solid Tumors (RECIST, version 1.1); one previous line of chemotherapy, including fluoropyrimidine or platinum, regardless of trastuzumab exposure; and adequate bone marrow and hepatic functions. Key exclusion criteria included: a history of hypersensitivity to drugs containing Cremophor® EL and trastuzumab; previous exposure to taxanes, and symptomatic central nervous system metastasis.

Patient Characteristics. A total of 44 patients (12 in phase I and 32 in phase II) were enrolled from 11 different treatment centers in Korea. The baseline characteristics of the study population are summarized in Table 1.

TABLE 1
Summary of Baseline Characteristics of Study Population

| | | | | Total |
|---|---|---|---|---|
| Phase I | | | Phase II | population |
| 8 mg | 12 mg | Total | RD: 8 mg | All |
| (n = 7) | (n = 5) | (n = 12) | (n = 32) | (n = 44) |

Age (years)

| | | | | | |
|---|---|---|---|---|---|
| Median (range) | 61.0 | 57.0 | 59.0 | 62.5 | 61.5 |
| | (41.0, 74.0) | (45.0, 73.0) | (41.0, 74.0) | (35.0, 76.0) | (35.0, 76.0) |

Sex, n(%)

| Male | 6 | (85.7) | 5 | (100.0) | 11 | (91.7) | 27 | (84.4) | 38 | (86.4) |
|---|---|---|---|---|---|---|---|---|---|---|
| Female | 1 | (14.3) | 0 | | 1 | (8.3) | 5 | (15.6) | 6 | (13.6) |
| ECOG PS, n(%) | | | | | | | | | | |
| 0 | 6 | (85.71) | 1 | (20.00) | 7 | (58.33) | 12 | (37.50) | 19 | (43.18) |
| 1 | 1 | (14.29) | 4 | (80.00) | 5 | (41.67) | 19 | (59.38) | 24 | (54.54) |

| | | | | | | | |
|---|---|---|---|---|---|---|---|
| 2 | — | — | — | 1 | (3.13) | 1 | (2.27) |

Metastasis at initial diagnosis, n(%)

| | | | | | | | | | | |
|---|---|---|---|---|---|---|---|---|---|---|
| Yes | 6 | (85.7) | 3 | (60.0) | 9 | (75.0) | 22 | (68.7) | 31 | (70.5) |
| No | 1 | (14.3) | 2 | (40.0) | 3 | (25.0) | 10 | (31.3) | 13 | (29.5) |

Primary tumor location at diagnosis, n(%)

| | | | | | | | | | | |
|---|---|---|---|---|---|---|---|---|---|---|
| Stomach | 7 | (100.0) | 5 | (100.0) | 12 | (100.0) | 30 | (93.7) | 42 | (95.4) |

| | | | | | | | |
|---|---|---|---|---|---|---|---|
| Gastroesophageal | 0 | 0 | 0 | 2 | (6.3) | 2 | (4.6) |
| junction | | | | | | | |

Location of the metastatic lesion at the screening of study

| | | | | | | | | | | |
|---|---|---|---|---|---|---|---|---|---|---|
| Lung | 2 | (28.6) | 1 | (20.0) | 3 | (25.0) | 9 | (28.1) | 12 | (27.3) |
| Lymph node | 6 | (85.7) | 4 | (80.0) | 10 | (83.3) | 21 | (65.6) | 31 | (70.5) |
| Liver | 5 | (71.4) | 3 | (60.0) | 8 | (66.7) | 20 | (62.5) | 28 | (63.6) |
| peritoneum | 1 | (14.3) | 1 | (20.0) | 2 | (16.7) | 13 | (40.6) | 15 | (34.1) |
| Bone | 1 | (14.3) | 1 | (20.0) | 2 | (16.7) | 2 | (6.3) | 4 | (9.1) |

| | | | | | | | |
|---|---|---|---|---|---|---|---|
| Brain | 0 | 0 | 0 | 1 | (3.1) | 1 | (2.3) |

Histologic type at diagnosis

| | | | | | | | | | | |
|---|---|---|---|---|---|---|---|---|---|---|
| Adenocarcinoma | 6 | (85.7) | 3 | (60.0) | 9 | (75.0) | 27 | (84.4) | 36 | (81.8) |

| | | | | | | | | | |
|---|---|---|---|---|---|---|---|---|---|
| SRCC | 1 | (14.3) | 0 | 1 | (8.3) | 3 | (9.4) | 4 | (9.1) |

| | | | | | | | | | |
|---|---|---|---|---|---|---|---|---|---|
| Mixed type | 0 | 2 | (40.0) | 2 | (16.7) | 1 | (3.1) | 3 | (6.8) |

| | | | | | | | |
|---|---|---|---|---|---|---|---|
| unknown | 0 | 0 | 0 | 1 | (3.1) | 1 | (2.3) |

HER2 status

| | | | | | | | | | | |
|---|---|---|---|---|---|---|---|---|---|---|
| IHC 3+ | 6 | (85.7) | 3 | (60.0) | 9 | (75.0) | 21 | (65.6) | 30 | (68.2) |
| IHC 2+ and FISH+ | 1 | (14.3) | 2 | (40.0) | 3 | (25.0) | 11 | (34.4) | 14 | (31.8) |

Previous gastrectomy

| | | | | | | | | | | |
|---|---|---|---|---|---|---|---|---|---|---|
| Yes | 3 | (42.9) | 1 | (20.0) | 4 | (33.3) | 9 | (28.1) | 13 | (29.5) |
| No | 4 | (57.1) | 4 | (80.0) | 8 | (66.7) | 23 | (71.9) | 31 | (70.5) |

Adjuvant chemotherapy

| | | | | | | | | | | |
|---|---|---|---|---|---|---|---|---|---|---|
| Yes | 1 | (14.3) | 1 | (20.0) | 2 | (16.7) | 7 | (21.9) | 9 | (20.5) |
| No | 6 | (85.7) | 4 | (80.0) | 10 | (83.3) | 25 | (78.1) | 35 | (79.5) |

Previous exposure of trastuzumab

| | | | | | | | | | | |
|---|---|---|---|---|---|---|---|---|---|---|
| Yes | 4 | (57.1) | 5 | (100.0) | 9 | (75.0) | 24 | (75.0) | 33 | (75.0) |

| | | | | | | | | | |
|---|---|---|---|---|---|---|---|---|---|
| No | 3 | (42.9) | 0 | 3 | (25.0) | 8 | (25.0) | 11 | (25.0) |

ECOG, Eastern Cooperative Oncology Group;
PS, performance status;
IHC, immunohistochemistry;
FISH, fluorescence in situ hybridization;
SRCC, signet ring cell carcinoma

This study was conducted in accordance with the Declaration of Helsinki and the International Conference on Harmonization Good Clinical Practice Guidelines and was approved by the Institutional Review Board of each participating center. All patients provided written informed consent before enrollment.

Treatment Procedures. Each 21-day cycle consisted of orally administered poziotinib once daily for 14 days, in combination with paclitaxel (175 mg/m2 infusion) and trastuzumab (8 mg/kg loading dose, followed by 6 mg/kg infusion) on day 1. In the phase I part of the study, increasing doses of poziotinib (8 mg, 12 mg or 16 mg) were tested to determine the RP2D of poziotinib, in combination with paclitaxel and trastuzumab. DLTs were evaluated in 6 patients at each poziotinib dose level. If DLT was observed in ≤1 patient, dose escalation was continued to the next dose level. If DLT was observed in ≥2 patients, dose escalation was stopped and the MTD was determined as the highest level at which DLT occurred in ≤1 patient. In the phase II part of the study, 32 patients were treated with the RP2D of poziotinib, in combination with paclitaxel and trastuzumab.

Safety Analyses and Efficacy. Adverse events (AEs) and TEAEs were evaluated using National Cancer Institute-Common Terminology Criteria for Adverse Events (version 4.03). DLTs were defined as grade z3 non-hematologic toxicities (except for alopecia); grade ≥3 diarrhea, nausea and vomiting, despite maximal dosage of anti-diarrhea and/or anti-emetic medication (as applicable); and grade 4 neutropenia sustained for ≥7 days, grade 3-4 neutropenia with fever or infection, grade 4 thrombocytopenia and grade 3 thrombocytopenia sustained for 7 days, or with accompanying bleeding, or requiring transfusion. Left ventricular ejection fraction (LVEF) assessment was performed at baseline, on day 1 of the first 3 cycles and every 3 cycles thereafter. All patients who received at least one dose of poziotinib were included in the safety analysis. Tumor response was evaluated by CT or MRI of the thorax, abdomen and pelvis every 6 weeks using RECIST version 1.1, according to investigator assessment.

Statistical Analysis. The primary objective of phase I was to assess the safety and tolerability of poziotinib and to determine the MTD of poziotinib when combined with paclitaxel and trastuzumab. The primary objective of the phase II part was to assess the objective response rate (ORR) of poziotinib combined with paclitaxel and trastuzumab. The secondary objectives were to evaluate the safety and tolerability, progression-free survival (PFS), time to tumor progression (TTP) and duration of overall response (DOR).

For phase II, it was hypothesized that an ORR <5% would be ineffective and an ORR >20% would be considered to be clinically significant. Sample size was calculated using Simon's 2-stage minimax design, with 80% power and a significance level of 5%. Of the total 27 target subjects, 13 were required in the 1^{st} stage. If the tumor response was 0 out of 13 patients, the study was planned to be terminated early. However, if at least 1 response was observed, an additional 14 patients would be enrolled in the study.

Results

PHASE I

Determination of the MTD. Seven patients were enrolled at dose level 1 (poziotinib 8 mg), however one patient was not evaluable for DLT due to loss to follow up before the completion of the first cycle. Among the 6 remaining patients, DLT was observed in 1 (Grade 4 neutropenia). At dose level 2 (poziotinib 12 mg), 5 patients were enrolled, and DLTs were observed in 2 (one with Grade 4 neutropenia and the other with febrile neutropenia with grade 4 neutropenia). Therefore, per the criteria outlined above, 8 mg poziotinib was determined to be the MTD.

Toxicity and dose adjustments. The safety analysis set consisted of 12 patients in phase I. TEAEs are summarized in Table 2. All 12 patients (100%) experienced ≥1 TEAEs with ≥Grade 3 toxicities observed in 11 patients (91.7%). TEAEs related to study discontinuation occurred in 3 patients (25.0%). TEAEs leading to death were reported in 1 patient (8.3%) (Table 2). The most common poziotinib-related AEs were diarrhea, rash, stomatitis, pruritus, and loss of appetite (Table 3). Grade 3 or higher neutropenia and febrile neutropenia were observed in 9 (75%) and 2 (16.7%) patients, respectively (Supplemental Table 1). Poziotinib dose reduction (from 8 to 6 mg) was performed for 1 (14.3%) of 6 patients. Dose reduction and discontinuation of paclitaxel was performed for 2 (28.6%) and 1 (14.3%) patients, respectively (Supplemental Table 2).

TABLE 2
Overall Summary of Treatment-Emergent Adverse Events (TEAEs)

| | | | | Total |
|---|---|---|---|---|
| Phase I | | | Phase II | population |
| 8 mg | 12 mg | Total | RD: 8 mg | All |
| (n = 7) | (n = 5) | (n = 12) | (n = 32) | (n = 44) |

Subjects with TEAEs, n (%)

| | | | | | | | | | |
|---|---|---|---|---|---|---|---|---|---|
| 7 | (100.0) | 5 | (100.0) | 12 | (100.0) | 32 | (100.0) | 44 | (100.0) |

| | | | | | |
|---|---|---|---|---|---|
| Exact 95% CI | 59.04-100.0 | 47.82-100.0 | 73.54-100.0 | 89.11-100.0 | 91.96-100.0 |

TEAEs by Maximum Severity, n (%)

| Grade 1 (mild) | 0 | | 0 | | 0 | | 0 | | 0 | |
|---|---|---|---|---|---|---|---|---|---|---|
| Grade 2 (moderate) | 1 | (14.3) | 0 | | 1 | (8.3) | 6 | (18.8) | 7 | (15.9) |
| Grade 3 (severe) | 2 | (28.6) | 1 | (20.0) | 3 | (25.0) | 20 | (62.5) | 23 | (52.3) |
| Grade 4 (life- | 3 | (42.9) | 4 | (80.0) | 7 | (58.3) | 3 | (9.4) | 10 | (22.7) |
| threatening) | | | | | | | | | | |

| | | | | | | | | | |
|---|---|---|---|---|---|---|---|---|---|
| Grade 5 (death) | 1 | (14.3) | 0 | 1 | (8.3) | 3 | (9.4) | 4 | (9.1) |

TEAEs leading to discontinuation and death

| | | | | | | | | | | |
|---|---|---|---|---|---|---|---|---|---|---|
| Discontinuation | 1 | (14.3) | 2 | (40.0) | 3 | (25.0) | 12 | (37.5) | 15 | (34.1) |

| | | | | | | | | | |
|---|---|---|---|---|---|---|---|---|---|
| Death | 1 | (14.3) | 0 | 1 | (8.3) | 3 | (9.4) | 4 | (9.1) |

TABLE 3
Safety Profiles: Adverse Events Related to Poziotinib (≥10% of patients)

| | Phase I | | | Phase II |
|---|---|---|---|---|
| | 8 mg | 12 mg | Total | RD: 8 mg |
| Safety Set | (n = 7) | (n = 5) | (n = 12) | (n = 32) |

Gastrointestinal disorder

| | | | | | | | | |
|---|---|---|---|---|---|---|---|---|
| Diarrhea | 7 | (100.0) | 5 | (100.0) | 12 | (100.0) | 29 | (90.6) |
| Stomatitis | 4 | (57.1) | 2 | (40.0) | 6 | (50.0) | 13 | (40.6) |
| Nausea | 1 | (14.3) | 1 | (20.0) | 2 | (16.7) | 3 | (9.4) |

| | | | | | |
|---|---|---|---|---|---|
| Vomiting | 0 | 0 | 0 | 4 | (12.5) |

Skin and subcutaneous tissue disorders

| | | | | | | | | |
|---|---|---|---|---|---|---|---|---|
| Rash | 4 | (57.1) | 3 | (60.0) | 7 | (58.3) | 22 | (68.8) |
| Pruritus | 1 | (14.3) | 1 | (20.0) | 2 | (16.7) | 7 | (21.9) |

| | | | | | | |
|---|---|---|---|---|---|---|
| Dermatitis acneiform | 2 | (28.6) | 0 | 2 | (16.7) | 0 |

Metabolism and nutrition disorders

| Decreased appetite | 0 | | 1 | (20.0) | 1 | (8.3) | 14 | (43.8) |
|---|---|---|---|---|---|---|---|---|
| Hypophagia | 0 | | 2 | (40.0) | 2 | (16.7) | 1 | (3.1) |
| Hypokalemia | 1 | (14.3) | 1 | (20.0) | 2 | (16.7) | 1 | (3.1) |

| | | | | | | | |
|---|---|---|---|---|---|---|---|
| Dehydration | 1 | (14.3) | 1 | (20.0) | 2 | (16.7) | 0 |

General disorders and administration site conditions

| | | | | | | | | |
|---|---|---|---|---|---|---|---|---|
| Mucosal inflammation | 1 | (14.3) | 2 | (40.0) | 3 | (25.0) | 3 | (9.4) |

| | | | | | |
|---|---|---|---|---|---|
| Fatigue | 0 | 0 | 0 | 7 | (21.9) |

Musculoskeletal and connective tissue disorders

| | | | | | |
|---|---|---|---|---|---|
| Myalgia | 0 | 0 | 0 | 4 | (12.5) |

Psychiatric disorders

| | | | | | | |
|---|---|---|---|---|---|---|
| Insomnia | 2 | (28.6) | 0 | 2 | (16.7) | 0 |

SUPPLEMENTAL TABLE 1
Incidence of Most Frequently Occurring (≥10%) and Grade ≥3 TEAEs Associated with Poziotinib

| | All Grade (≥10%) | | | | | | | | ≥Grade 3 | | | | | | | |
|---|---|---|---|---|---|---|---|---|---|---|---|---|---|---|---|---|
| | Phase 1 | | | | | | | | Phase 1 | | | | | | | |
| Preferred | 8 mg | | 12 mg | | Total | | Phase 2 | | 8 mg | | 12 mg | | Total | | Phase 2 | |
| term (%) | (n = 7) | | (n = 5) | | (n = 12) | | n = 32 | | (n = 7) | | (n = 5) | | (n = 12) | | n = 32 | |
| Subjects with | 7 | (100.0) | 5 | (100.0) | 12 | (100.0) | 32 | (100.0) | 6 | (85.7) | 5 | (100.0) | 11 | (91.7) | 26 | (81.3) |
| TEAEs | | | | | | | | | | | | | | | | |
| Diarrhea | 7 | (100.0) | 5 | (100.0) | 12 | (100.0) | 30 | (93.8) | 1 | (14.3) | 3 | (60.0) | 4 | (33.3) | 13 | (40.6) |
| Stomatitis | 4 | (57.1) | 2 | (40.0) | 6 | (50.0) | 16 | (50.0) | 0 | | 0 | | 0 | | 1 | (3.13) |
| Vomiting | 2 | (28.6) | 2 | (40.0) | 4 | (33.3) | 8 | (25.0) | 0 | | 1 | (20.0) | 1 | (83.3) | 0 | |
| Abdominal | 1 | (14.3) | 0 | | 1 | (8.3) | 11 | (34.4) | 0 | | 0 | | 0 | | 2 | (6.3) |
| pain | | | | | | | | | | | | | | | | |
| Hypokalemia | 2 | (28.6) | 3 | (60.0) | 5 | (41.7) | 6 | (15.6) | 1 | (14.3) | 3 | (60.0) | 4 | (33.3) | 1 | (3.13) |
| Decreased | 1 | (14.3) | 1 | (20.0) | 2 | (16.7) | 19 | (59.4) | 1 | (14.3) | 0 | | 1 | (8.3) | 5 | (15.6) |
| appetite | | | | | | | | | | | | | | | | |
| Hypophagia | 0 | | 3 | (60.0) | 3 | (25.0) | 0 | | 1 | (14.3) | 1 | (20.0) | 2 | (16.7) | 0 | |
| Rash | 4 | (57.1) | 3 | (60.0) | 7 | (58.3) | 22 | (68.8) | 0 | | 0 | | 0 | | 4 | (12.5) |
| Pruritus | 1 | (14.3) | 2 | (40.0) | 3 | (25.0) | 7 | (21.9) | 0 | | 0 | | 0 | | 1 | (3.1) |
| Mucosal | 1 | (14.3) | 2 | (40.0) | 3 | (25.0) | 3 | (9.4) | 0 | | 0 | | 0 | | 2 | (6.3) |
| inflammation | | | | | | | | | | | | | | | | |
| Neutropenia | 4 | (57.1) | 5 | (100.0) | 9 | (75.0) | 4 | (12.5) | 4 | (67.1) | 5 | (100.00) | 9 | (75.0) | 4 | (12.6) |
| Anemia | 2 | (28.6) | 3 | (60.0) | 5 | (41.7) | 12 | (37.5) | 2 | (28.6) | 2 | (40.0) | 4 | (33.3) | 7 | (21.9) |
| Febrile | 1 | (14.3) | 1 | (20.0) | 2 | (16.7) | 3 | (9.4) | 1 | (14.3) | 1 | (20.0) | 2 | (16.7) | 3 | (9.4) |
| neutropenia | | | | | | | | | | | | | | | | |

| | | | | | | | | | | | | | | |
|---|---|---|---|---|---|---|---|---|---|---|---|---|---|---|
| Pneumonia | 0 | 2 | (40.0) | 2 | (16.7) | 5 | (15.6) | 0 | 1 | (20.0) | 1 | (8.3) | 4 | (12.5) |

| | | | | | | | | | | | | | | |
|---|---|---|---|---|---|---|---|---|---|---|---|---|---|---|
| Hypotension | 2 | (28.6) | 0 | 2 | (16.7) | 1 | (3.1) | 2 | (28.6) | 0 | 2 | (16.7) | 1 | (3.1) |

SUPPLEMENTAL TABLE 2
Dose Adjustments

| Phase I | | | Phase II |
|---|---|---|---|
| 8 mg | 12 mg | Total | RD: 8 mg |
| (n = 71) | (n = 5) | (n = 12) | (n = 32) |

Poziotinib (mg), n(%)

| | | | | | | | |
|---|---|---|---|---|---|---|---|
| 8 (starting dose) | 5 | (71.4) | — | 5 | (41.7) | 10 | (31.3) |
| 6 | 1 | (14.3) | — | 1 | (8.3) | 14 | (43.8) |

| | | | | |
|---|---|---|---|---|
| 4 | — | — | 6 | (18.8) |

| | | | | | | |
|---|---|---|---|---|---|---|
| 12 (starting dose) | — | 1 | (20.0) | 1 | (8.3) | — |
| 8 | — | 4 | (80.0) | 4 | (33.3) | — |

Paclitaxel (mg/m2), n(%)

| 175 | 3 | (42.9) | — | | 3 | (25.0) | 13 | (40.6) |
|---|---|---|---|---|---|---|---|---|
| 150 | — | | 1 | (20.0) | 1 | (8.3) | 6 | (18.8) |
| 125 | 2 | (28.6) | — | | 2 | (16.7) | 5 | (15.6) |
| 105 | — | | — | | | | 1 | (3.1) |
| Discontinuation | 1 | (14.3) | 4 | (80.0) | 5 | (41.7) | 3 | (9.4) |

Efficacy. A total of 11 patients were available for the evaluation of the efficacy of poziotinib. The objective response was observed in four patients (33.3%; 95% Cl, 9.9-65.1)

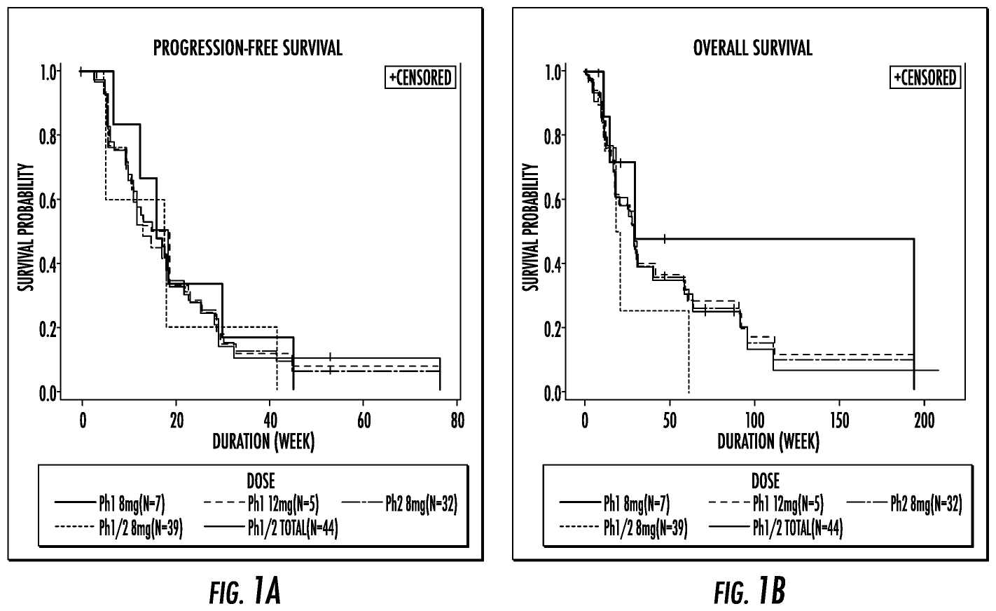

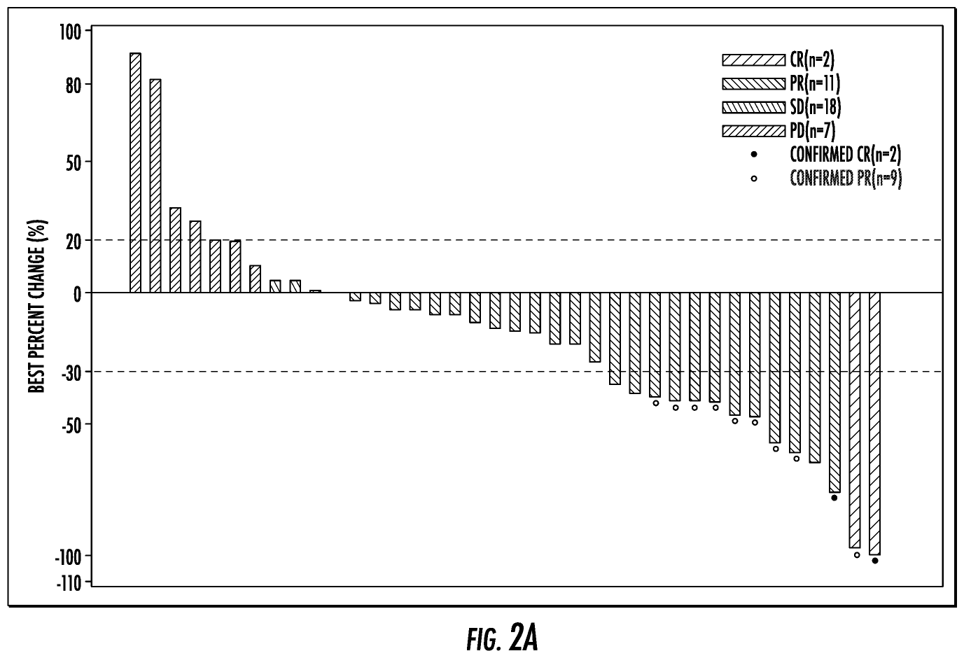

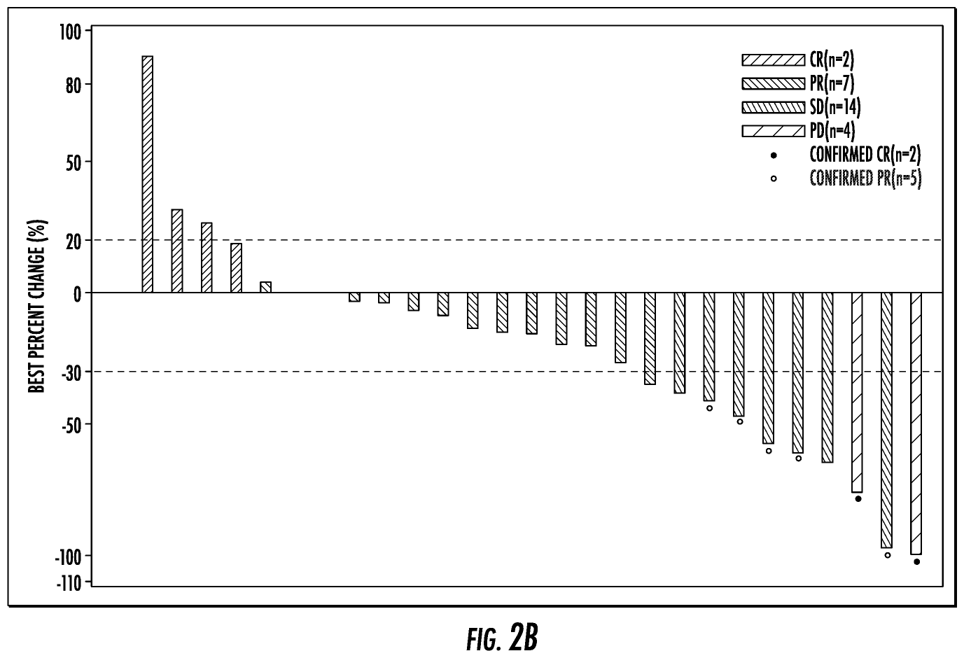

(Table 4). All four had partial response. The disease control rate (DCR; PR+SD) was 66.7% (95% Cl, 34.9-90.1). Median PFS and OS were 17,7 weeks (95% CI, 5.4-30.2) and 30.6 weeks (95% Cl, 12.2-195.0) respectively (Table 5 and FIG. 1).

TABLE 4
Summary of Tumor Responses

| | | | | | | | | | Total | |
|---|---|---|---|---|---|---|---|---|---|---|
| | Phase I | | | | | | Phase II | | population | |
| | 8 mg | | 12 mg | | Total | | RD: 8 mg | | All | |
| | (n = 7) | | (n = 5) | | (n = 12) | | (n = 32) | | (n = 44) | |
| Objective response rate, n(%) | 2 | (28.6) | 2 | (40.0) | 4 | (33.3) | 7 | (21.9) | 11 | (25.0) |
| Exact 95% CI | 3.7-71.0 | | 5.3-85.3 | | 9.9-65.1 | | 9.3-40.0 | | 13.2-40.3 | |
| Disease control rate, n(%) | 5 | (71.4) | 3 | (60.0) | 8 | (66.7) | 23 | (71.9) | 31 | (70.5) |

| | | | | | |
|---|---|---|---|---|---|
| Exact 95% CI | 29.0-96.3 | 14.7-94.7 | 34.9-90.1 | 53.3-86.3 | 54.8-83.2 |

Best overall response, n(%)

| CR | 0 | | 0 | | 0 | | 2 | (6.3) | 2 | (4.5) |
|---|---|---|---|---|---|---|---|---|---|---|
| PR | 2 | (28.6) | 2 | (40.0) | 4 | (33.3) | 5 | (15.6) | 9 | (20.5) |
| SD | 3 | (42.9) | 1 | (20.0) | 4 | (33.3) | 16 | (50.0) | 20 | (45.5) |
| PD | 1 | (14.3) | 2 | (40.0) | 3 | (25.0) | 5 | (15.6) | 8 | (18.2) |
| unknown | 1 | (14.3) | 0 | | 1 | (8.3) | 4 | (12.5) | 5 | (11.4) |
| Trastuzumab-pretreated | 4 | | 5 | | 9 | | 24 | | 33 | |
| patients, n | | | | | | | | | | |
| Objective response rate, n (%) | 1 | (25.0) | 2 | (40.0) | 3 | (33.3) | 3 | (12.5) | 6 | (18.2) |
| Exact 95% CI | 0.6-80.6 | | 5.3-85.3 | | 7.5-70.1 | | 2.7-32.4 | | 7.1-35.5 | |
| Disease control rate, n (%) | 3 | (75.0) | 3 | (60.0) | 6 | (66.7) | 18 | (75.0) | 24 | (72.7) |

| Exact 95% CI | 19.4-99.46 | | 14.7-94.7 | 29.9-92.5 | | 53.3-90-2 | | 54.5-86.7 | |
|---|---|---|---|---|---|---|---|---|---|
| Trastuzumab-naïve patients, n | 3 | | 0 | 3 | | 8 | | 11 | |
| Objective response rate, n (%) | 1 | (33.3) | 0 | 1 | (33.3) | 4 | (50.0) | 5 | (45.5) |

| | | | | |
|---|---|---|---|---|
| Exact 95% CI | 0.8-90.6 | 0.8-90.6 | 15.7-84.3 | 16.8-76.6 |

| | | | | | | | | | |
|---|---|---|---|---|---|---|---|---|---|
| Disease control rate, n (%) | 2 | (66.7) | 0 | 2 | (66.7) | 5 | (62.5) | 7 | (63.6) |

| | | | | |
|---|---|---|---|---|
| Exact 95% CI | 9.4-99.2 | 9.4-99.2 | 24.5-91.5 | 30.8-89.1 |

TABLE 5
Summary of survival data

| | | | | | Total |
|---|---|---|---|---|---|
| | Phase I | | | Phase II | population |
| | 8 mg | 12 mg | Total | RD: 8 mg | Total |
| All patients | 7 | 5 | 12 | 32 | 44 |

Progression free survival (weeks)

| | | | | | |
|---|---|---|---|---|---|
| Median | 17.1 | 17.7 | 17.7 | 13.0 | 16.0 |
| Exact 95% CI | 7.0-45.3 | 5.3-41.6 | 5.4-30.2 | 9.8-21.9 | 10.7-18.4 |

Overall survival (weeks)

| | | | | | |
|---|---|---|---|---|---|
| Median | 30.6 | 19.9 | 30.6 | 29.5 | 29.5 |
| Exact 95% CI | 12.2-195.0 | 11.8-61.7 | 12.2-195.0 | 17.9-59.2 | 18.7-59.2 |

Time to tumor progression (weeks)

| | | | | | |
|---|---|---|---|---|---|
| Median | 18.2 | 17.7 | 17.9 | 15.0 | 17.1 |
| Exact 95% CI | 7.0-45.3 | 5.3-41.6 | 5.4-30.2 | 10.0-23.1 | 11.1-21.9 |

Duration of overall response (weeks)*

| | | | | | |
|---|---|---|---|---|---|
| Median | 14.5 | 20.4 | 15.0 | 26.8 | 20 1 |
| Exact 95% CI | 10.5-18.4 | 11.7-29.0 | 10.5-29.0 | 17.2-71.8 | 11.7-71.8 |
| Trastuzumab- | 4 | 5 | 9 | 24 | 33 |
| pretreated Patients, n | | | | | |

Progression free survival (weeks)

| | | | | | |
|---|---|---|---|---|---|
| Median | 15.5 | 17.7 | 17.7 | 13.0 | 15.0 |
| Exact 95% CI | 7.0-30.2 | 5.3-41.6 | 5.3-30.2 | 9.8-18.7 | 10.0-18.4 |

Overall survival (weeks)

| | | | | | |
|---|---|---|---|---|---|
| Median | 30.6 | 19.9 | 21.1 | 29.5 | 29.5 |
| Exact 95% CI | 12.2-30.6 | 11.8-61.7 | 11.8-61.7 | 17.9-40.9 | 18.7-31.3 |
| Trastuzumab-naïve | 3 | 0 | 3 | 8 | 11 |
| patients, n | | | | | |

Progression free survival (weeks)

| | | | | | |
|---|---|---|---|---|---|
| Median | 30.6 | — | 30.6 | 18.7 | 20.7 |
| Exact 95% CI | 16.0-45.3 | | 16.0-45.3 | 3.0− | 3.0-45.3 |

Overall survival (weeks)

| | | | | | |
|---|---|---|---|---|---|
| Median | 194.9 | — | 194.9 | 42.6 | 59.2 |
| Exact 95% CI | 16.0-194.9 | | 16.0-194.9 | 3.0-111.5 | 10.7-194.9 |

CI, confidence interval. If the last administration date was not collected, the End of Study Date was used. The numbers of patients in 8 mg and 12 mg in phase I were 2 and 2. And the number of patients in phase II (8 mg) was 7. A total of 11 were calculated in terms of duration of overall response.

PHASE II—See tables above.

A total of 32 patients were included in phase II. Twenty patients (63%) received a reduced dose of poziotinib. See Supplemental Table 2, above.

Safety analysis. Grade 3 or higher TEAEs neutropenia and febrile neutropenia developed in 4 (12.5%) and 3 (9.4%) of patients, respectively (Supplemental Table 1). The most common poziotinib-related TEAEs were diarrhea, rash, stomatitis and decreased appetite, similar to results obtained in phase I (Table 3). Grade 3 or higher diarrhea was observed in 13 patients (40.6%) (Supplemental Table 1). Four patients (12.5%) discontinued the study before the first assessment due to treatment-related toxicity. There was no grade 3 or 4 left ventricular systolic dysfunction (LVSD). Twenty (62.5%) patients received a reduced dose of poziotinib. A reduced dose of paclitaxel was administered to 12 patients (37.5%) and paclitaxel was discontinued in 3 patients (9.4%) (Supplemental Table 2)

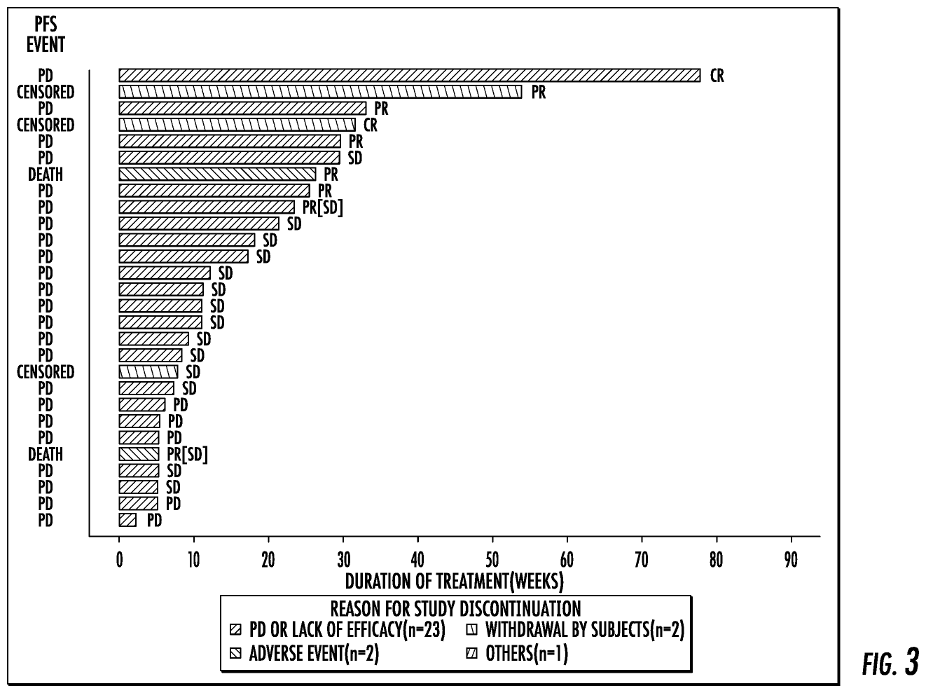

Efficacy. Twenty patients (62.5%) had tumor shrinkage (FIG. 1). Confirmed response was observed in 7 patients (21.9%; 95% CI, 9.3-40.0). Two patients (6.3%) had complete responses and 5 (15.6%) had partial responses. DCR was 71.9% (95% Cl, 53.3-86.3) (Table 4). The median PFS was 13.0 weeks (95% Cl, 9.8-21.9) (Table 5, FIGS. 1 and 3). The median OS was 29.5 weeks (95% Cl, 17.9-59.2) (Table 5, FIG. 1). The median TTP and DOR were 15.0 weeks (95% Cl, 10.0-23.1) and 26.8 weeks (95% Cl, 17.2-71.8), respectively (Table 5). When looking into clinical efficacy in terms of trastuzumab exposure, the ORRs of trastuzumab-pretreated and trastuzumab-naïve patients were 12.5% and 50.0%, respectively (Table 4). The median OS and PFS were 29.5 weeks (95% Cl, 17.9-40.9) and 13.0 weeks (9.8-18.7) in trastuzumab-pretreated patients, and 42.6 weeks (95% Cl, 3.0-111.5) and 18.7 weeks (95% Cl, 3.0-) in trastuzumab-naïve patients (Table 5).

Discussion

In this prospective, multicenter, open-label phase I/II study, poziotinib 8 mg combined with paclitaxel plus trastuzumab showed manageable toxicity and promising efficacy in previously-treated patients with advanced HER2-positive gastric cancer (GC). To our knowledge, this study is the first to evaluate a pan-HER inhibitor combined with a chemotherapeutic agent and trastuzumab in HER2-positive tumors.

In a previous phase I study, poziotinib MTDs were determined to be 24 mg/day with intermittent dosing (14 days on and 7 days off) and 18 mg/day with continuous dosing . In the phase I part of the current study, the MTD of poziotinib was determined to be 8 mg/day, which is much lower than the poziotinib MTD of 24 mg/day as monotherapy. Commonly observed TEAEs in our study were diarrhea, rash, stomatitis and pruritus. All patients experienced at least one TEAE in this study, with a frequency similar to that reported in the phase I trial of poziotinib monotherapy. In contrast to the phase I monotherapy trial, all DLTs in our study were reported to be grade 4 neutropenia or febrile neutropenia accompanying grade 4 neutropenia, rather than diarrhea.

In the aforementioned phase I trial, the ORR and median PFS of poziotinib monotherapy was reported to be 16% and 12.0 weeks (intermittent dosing schedule) and 21% and 9.0 weeks (continuous dosing schedule). Another pan-HER inhibitor, dacomitinib, as monotherapy produced an ORR of 7.4% (95% Cl, 0-17.5) and a median PFS of 2.1 months (95% Cl, 2.3-3.4) in HER2-positive GC. Given the ORR and median PFS of our study, these results indicate that pan-HER inhibitors, in combination with chemotherapy, could be more potent than pan-HER inhibitors alone.

Dual blockade of HER2 receptors has become the standard of care in HER2-positive breast cancer. Pertuzumab in combination with docetaxel plus trastuzumab significantly increased OS in HER2-positive breast cancer. However, in contrast to HER2-positive breast cancer, pertuzumab in combination with chemotherapy did not extend OS in HER2-positive GC. This finding indicates that the efficacy of dual blockade in HER2-positive GC might be different from that in HER2-positive breast cancer. However, even in vitro, dual blockade with lapatinib and trastuzumab showed highly synergistic anti-tumor activity against HER2-amplified GC cells. In the current study, poziotinib in combination with trastuzumab showed a good tumor response, indicating that this dual blockade strategy, including pan-HER inhibitors, would be a promising treatment option in HER2-positive GC as salvage treatment.

The continuation of anti-HER2 targeted therapy beyond progression is a generally accepted treatment strategy in HER2-positive breast cancer. A retrospective study reported that trastuzumab beyond progression increased PFS and OS in HER2-positive GC. However, in a prospective phase II study, trastuzumab beyond progression as second-line failed to improve PFS and the ORR in patients with HER2-positive GC, indicating that the role of trastuzumab beyond progression remains controversial in HER2-positive GC.

Antibody-drug conjugate (ADC) is one of the emerging treatment strategies in HER2-positive tumors. A phase III trial of trastuzumab emtansine was performed in patients with HER2-positive GC, who failed prior fluoropyrimidine and platinum. However, trastuzumab emtansine was not superior to paclitaxel. The ORR was only 20.6% (95% Cl, 15.26-26.45). DS8201a, a new HER2-targeting ADC, has shown potent anti-tumor'activity against HER2-positive GC cells. In a phase I study, DS8201a presented an ORR of 44% and DCR of 78% in patients with HER2-poisitive GC resistant to trastuzumab, and also in patients with low HER2-expressing GC, indicating that HER2-targeting ADC would be a promising salvage treatment in HER2-positive GC).

In the current study, pretreatment biopsy was not mandatory, therefore patients were enrolled based on HER2 expression at initial diagnosis. However, differences in HER2 expression before and after trastuzumab were reported in HER2-positive GC and a loss of HER2 positivity was even observed in 32% of patients who received trastuzumab. These observations indicate that re-assessment of exact HER2 status is essential just before re-challenge with anti-HER2 targeted therapy and, as such, this represents a limitation of our study.

In conclusion, poziotinib (8 mg) in combination with paclitaxel and trastuzumab showed good clinical efficacy and manageable toxicity in patients with HER2-positive GC who had received one line of chemotherapy. Dual blockade inhibition using trastuzumab with a pan-HER inhibitor is a promising strategy to overcome resistance to trastuzumab.

It should be understood that embodiments described herein should be considered as illustrative only, without limiting the scope of the invention. Descriptions of features or aspects within each embodiment should typically be considered as available for other similar features or aspects in other embodiments. It will be understood by those of ordinary skill in the art that various changes in form and details may be made therein without departing from the spirit and scope of the disclosure as defined by the following claims.

1. A method of treating cancer in a subject, the method comprising administering to the subject therapeutically effective amounts of poziotinib and an anti-HER1, anti-HER2 or anti-HER4 antibody, cwherein the cancer is associated with overexpression or amplification of HER1, HER2, or HER4, or a mutant of HER1, HER2, or HER4, and
wherein said cancer is non-small cell lung cancer, breast cancer, colon cancer, gastric cancer, pancreatic cancer, prostate cancer, myeloma, head cancer, neck cancer, ovarian cancer, esophageal cancer, or metastatic cell carcinoma.

2. The method of claim 1, further comprising administering at least one agent selected from the group consisting of paclitaxel, cisplatin, 5-fluorouracil, vinorelbine, cetuximab and any combinations thereof.

3. The method of claim 1, wherein said antibody is an anti-HER2 antibody selected from the group consisting of trastuzumab, cetuximab, and any antigen-binding fragments thereof.

4. The method of claim 1, wherein the cancer is breast cancer.

5. The method of claim 4, wherein said breast cancer is selected from the group consisting of:
(i) estrogen receptor-negative breast cancer with overexpression of HER1 and/or HER2;
(ii) estrogen receptor- and progesterone receptor-double positive breast cancer with HER2 being expressed but without overexpression thereof;
(iii) trastuzumab-resistant breast cancer with overexpression of HER2; and
(iv) HER1-overexpressing breast cancer negative with respect to PR, HER2 and estrogen receptor.

6. The method of claim 5, wherein said breast cancer is metastatic breast cancer.

7. The method of claim 6, further comprising the preliminary steps of:
collecting breast cancer cells from said subject; and
evaluating said breast cancer cells to confirm overexpression of HER2, or
overexpression of a mutant of HER2, or amplification of the HER2 gene, or
amplification of a mutant of the HER2 gene.

8. The method of claim 7, wherein said evaluating step comprises immunohistochemistry (IHC) with confirmatory fluorescence in situ hybridization (FISH).

9. The method of claim 8 wherein said IHC is IHC 3+ or IHC 2+.

10. A method of treating breast cancer in a subject in need thereof, wherein said breast cancer is associated with overexpression or amplification of HER2, or overexpression or amplification of a mutant of HER2, said method comprising the steps of:
a) in a 21-day cycle±3 days:
i) administering a single dose of T-DM1 ranging from 1.5-5.5 mg/kg;
ii) administering a daily dose of poziotinib ranging from 0.5-50 mg/day; and
iii) optionally resting from treatment for a period of time within the 21-day cycle; and
b) optionally repeating said cycle, or a variation of said cycle.

11. The method of claim 10, wherein said 21-day cycle comprises 2 weeks of drug administering with a 1-week resting period.

12. The method of claim 11, wherein said 21-day cycle is selected from the group consisting of
two weeks of administering followed by one week of resting;
one week of administering, followed by one week of resting, followed by another week of administering; and
one week of resting followed by two weeks of administering.

13. The method of claim 10, wherein T-DM1 is administered by intravenous (IV) infusion.

14. The method of claim 13, wherein the T-DM1 standard dose is 3.6 mg/kg.

15. The method of claim 10, wherein poziotinib is administered orally.

16. The method of claim 15, wherein the oral dose is selected from the group consisting of 6, 8, 10, 12, 16 and 24 mg once per day.

17-19. (canceled)

20. A method of treating gastric cancer in a subject, the method comprising administering to the subject therapeutically effective amounts of poziotinib and an anti-HER1, anti-HER2 or anti-HER4 antibody,
wherein the gastric cancer is associated with overexpression or amplification of HER1, HER2, or HER4, or a mutant of HER1, HER2, or HER4.

21. The method of claim 21, further comprising administering at least one agent selected from the group consisting of paclitaxel, cisplatin, 5-fluorouracil, vinorelbine, cetuximab and any combinations thereof.

22. The method of claim 20, wherein said antibody is an anti-HER2 antibody selected from the group consisting of trastuzumab, cetuximab, and any antigen-binding fragments thereof.

23. The method of claim 20, further comprising the preliminary steps of:
collecting gastric cancer cells from said subject; and
evaluating said gastric cancer cells to confirm overexpression of HER2, or
overexpression of a mutant of HER2, or amplification of the HER2 gene, or
amplification of a mutant of the HER2 gene.

24. The method of claim 20, wherein said evaluating step comprises immunohistochemistry (IHC) with confirmatory fluorescence in situ hybridization (FISH).

25. The method of claim 24 wherein said IHC is IHC 3+ or IHC 2+.

26. The method of claim 20, wherein said gastric cancer has been previously treated with a chemotherapy regimen.

27. A method of treating gastric cancer in a subject in need thereof, wherein said gastric cancer is associated with overexpression or amplification of HER2, or overexpression or amplification of a mutant of HER2, said method comprising the steps of:
a) in a 21-day cycle±3 days:
i) administering a single dose of trastuzumab ranging from 6-8 mg/kg;
ii) administering a single dose of paclitaxel ranging from 105-175 mg/m²; and
iii) administering a daily dose of poziotinib ranging from 4-16 mg/day; and
b) optionally repeating said cycle.

28. The method of claim 27, wherein trastuzumab is administered by intravenous (IV) infusion on Day 1.

29. The method of claim 27 [[or 28]], wherein the trastuzumab standard dose is 8 mg/kg loading followed by 6 mg/kg infusion.

30. The method of claim 27, wherein the paclitaxel is administered by intravenous (IV) infusion on Day 1.

31. The method of claim 30, wherein the paclitaxel standard dose is 175 mg/m² infusion.

32. The method of claim 27, wherein poziotinib is administered orally for 14±3 days.

33. The method of claim 32, wherein the oral dose is selected from the group consisting of 4, 6, 8, 10, 12, and 16 mg once per day.

34. The method of claim 33, wherein a rest period of 7 days follows the 14-day poziotinib administration.

35-37. (canceled)
